(19) United States
(12) Patent Application Publication
(10) Pub. No.: US 2020/0353122 A1
(43) Pub. Date: Nov. 12, 2020

(54) COLLAGEN-FIBRIN COMPOSITION, METHOD AND WOUND ARTICLES

(71) Applicant: 3M INNOVATIVE PROPERTIES COMPANY, St. Paul, MN (US)

(72) Inventors: Bryan A. Baker, Minneapolis, MN (US); Jason W. Bjork, Cottage Grove, MN (US)

(21) Appl. No.: 16/764,433

(22) PCT Filed: Nov. 15, 2018
(86) PCT No.: PCT/IB2018/059005

§ 371 Date: May 15, 2020

Related U.S. Application Data
(60) Provisional application No. 62/592,182, filed on Nov. 29, 2017.

(51) Int. Cl. A61L 26/00

(52) U.S. Cl. CPC A61L 26/0052; A61L 26/008; A61L 26/0085; A61L 26/0042; A61L 26/0033

(57) ABSTRACT

A method is described. The method includes mixing fibrin and collagen to form a mixture including collagen and fibrin; and reducing the salt concentration below the threshold concentration to form a fibrin. A collagen-fibrin composition is also described. The composition includes a collagen and a fibrin; wherein the composition has a salt concentration below the threshold concentration to form a fibrin.

### BACKGROUND

Fibrinogen is cleaved and polymerized into fibrin using thrombin in a well-characterized process. Thrombin cleaves fibrinogen, forming fibrin monomers. Once fibrinogen is cleaved, fibrin monomers come together and form a covalently crosslinked fibrin network in the presence of factors, such as Factor XIII, normally present in blood. At a wound site, the fibrin network helps to close the wound and promote healing.

Collagen dressings are used as wound care products. These products are primarily derived from bovine collagen sources, particularly bovine skin, and processed via acid or enzymatic extraction methods into purified and largely type I collagen material. There is a need to provide better collagen dressings in a form useful for treating wounds.

### SUMMARY

In one embodiment, a method is described. The method includes mixing fibrin and collagen to form a mixture comprising of collagen and fibrin; and reducing the salt concentration below the threshold concentration to form a fibrin.

In another embodiment, a method is described. The method includes mixing collagen, fibrinogen and fibrin-forming enzyme to form a mixture comprising of collagen and fibrin; and reducing the salt concentration below the threshold concentration to form a fibrin.

In another embodiment, a composition is described. The composition includes a collagen and a fibrin; wherein the composition has a salt concentration below the threshold concentration to form a fibrin.

In another embodiment, a wound dressing comprising the composition of the current application is described.

### BRIEF DESCRIPTION OF THE DRAWINGS

FIG. 1 is a schematic cross-section of an illustrative embodiment of a collagen-fibrin article suitable for a wound dressing comprising a collagen-fibrin composition in the form of a sheet;

FIG. 2 is a schematic cross-section of an illustrative embodiment of a collagen-fibrin article suitable for a wound dressing comprising a collagen-fibrin composition in the form of a foam;

FIG. 3 is a schematic cross-section of an illustrative embodiment collagen-fibrin article suitable for a wound dressing comprising a sheet of foam and collagen-fibrin particles;

FIG. 4 is a schematic cross-section of an illustrative embodiment of a collagen-fibrin article suitable for a wound dressing comprising a collagen-fibrin containing layer and a carrier layer;

FIG. 5 is a Scanning Electron Microscopy (SEM) image of lyophilized collagen-fibrin article.

### DETAILED DESCRIPTION

In some embodiments, a method of forming a collagen-fibrin composition is described. As used herein, “fibrin” refers to a protein formed by the reaction of fibrinogen with a fibrin-forming enzyme (e.g. thrombrin). Such enzyme is capable of cleaving fibrin A and B peptides from fibrinogen and convert it to fibrin. Fibrinogen is a precursor to fibrin.

The method comprises mixing fibrin and collagen to form a mixture comprising of collagen and fibrin. The fibrin can be formed by mixing fibrinogen and fibrin-forming enzyme to form an aqueous solution comprising fibrinogen, a fibrin-forming enzyme and salt. Alternatively, in other embodiments, collagen, fibrinogen and fibrin-forming enzyme can be mixed to form the mixture comprising of collagen and fibrin. Fibrin or the mixture of collagen and fibrin can be in a form of gel, for example, hydrogel.

Thrombin is the most common fibrin-forming enzyme. Alternative fibrin-forming enzymes include batroxobin, crotalase, ancrod, reptilase, gussurobin, recombinant thrombin-like enzymes, as well as venom of 20 to 30 different species of snakes. The fibrin-forming enzyme can be any one or combination of such fibrin-forming enzymes.

Any suitable sources of collagen, fibrinogen and thrombin can be used in the preparation of the mixture comprising of collagen and fibrin (e.g. collagen-fibrin gel composition). For example, the species from which the collagen is obtained could be human, bovine, porcine, or other animal sources. Similarly, fibrinogen and thrombin can also be obtained from human, bovine, porcine, or other animal sources. Collagen, fibrinogen and thrombin can also be obtained from recombinant sources. Collagen, fibrinogen and thrombin can also be obtained commercially as aqueous solutions, and the concentrations of these solutions may vary. Alternatively, collagen, fibrinogen and thrombin can be provided in lyophilized form and stored at very low temperatures. Lyophilized fibrinogen is typically reconstituted with sterile water before use to form an aqueous solution. Thrombin is also reconstituted with sterile calcium chloride and water before use. Saline, phosphate buffered solution, or other reconstituting liquid can also be used. In preparing fibrin, the reconstituted fibrinogen and thrombin are then combined to form fibrin. In some embodiments, collagen or fibrin can be dissolved in acetic acid.

In some embodiments, the amount of collagen is at least 1 mg/mL and typically no greater than 120 mg/mL. The amount of fibrinogen and fibrin-forming enzyme (e.g. thrombin) can be sufficient to produce the desired amount of fibrin. In some embodiments, the amount of fibrinogen is at least 1 mg/mL, at least 5 mg/mL, at least 10 mg/mL and typically no greater than 120, 100, 75, 50 mg/mL. In some embodiments, the amount of fibrinogen is no greater than 75, 50, 25, 20, 15, 10 or 5 mg/mL. Further, the amount of fibrin-forming enzyme (e.g. thrombin) is at least 0.01, 0.02, 0.03, 0.04, or 0.05 Units/milliliter (U/mL) and typically no greater than 500 U/mL. In some embodiments, the amount of fibrin-forming enzyme (e.g. thrombin) is no greater than 250, 125, 50, 25, 20, 15, 10, or 5, 4, 3, 2, or 1 U/mL. Aqueous solutions of fibrinogen typically comprise salt (e.g. saline). The salt concentration is sufficient such that the fibrinogen forms a solution. Alternatively, solid fibrinogen can be reconstituted in saline or other salt solution. In a typical embodiment, substantially all the fibrinogen is converted to fibrin. Excess fibrin-forming enzyme (e.g. thrombin) is removed when the fibrin hydrogel is rinsed to reduce the salt content.

The aqueous solution further comprises salt suitable for producing a fibrin containing hydrogel. Thus, such salt can be characterized as a fibrin hydrogel forming salt. The fibrin is generally uniformly dispersed and soluble in the hydrogel. Hence, the hydrogel typically contains little or no fibrin precipitates. When a fibrin hydrogel is formed, the hydrogel is generally a continuous two-phase system that can be handled as a single mass.

Various salts with Group I and/or Group II metal cations have been utilized to solubilize protein such as potassium, sodium, lithium, magnesium, and calcium. Other cations utilized in protein synthesis include ammonium and guanidinium.

Various anions have also been utilized to solubilize protein. Although chloride anion is most common, nitrate and acetate are most similar to chloride according to the Hofmeister series, i.e. a classification of ions in order of their ability to salt out (e.g. precipitate) or salt in (e.g. solubilize) proteins.

In some embodiments, the salt comprises sodium chloride. The amount of sodium chloride in the aqueous solution and fibrin hydrogel, prior to dehydration, is typically greater than 0.09 wt.-% of the solution. The concentration of sodium chloride may be at least 0.10, 0.20, 0.30, 0.04, 0.50, 0.60, 0.70, 0.80 or “normal saline” 0.90 wt.-% and typically no greater than 1 wt.-%. Minimizing the salt concentration is amenable to minimizing the salt that is subsequently removed.

The salt typically comprises a calcium salt, such as calcium chloride. The amount of calcium salt in the aqueous solution and fibrin hydrogel, prior to dehydration, is typically at least 0.0015%, 0.0020%, or 0.0030% wt.-% and typically no greater than 0.5 wt.-%.

In typical embodiments, a buffering agent is also present to maintain the desired pH range. In some embodiments, the pH ranges from 6 to 8 or 7 to 8 during the formation of the fibrin. Various buffering agent are known. Buffering agents are typically weak acids or weak bases. One suitable buffering agent is a zwitterionic compound known as HEPES (4-(2-hydroxyethyl)-1-piperazineethanesulfonic acid). Other buffering agents, such as those commonly known as Good buffers can also be utilized. In some embodiments, the buffering agent does not substantially contribute to the formation of the fibrin hydrogel. For example when the salt contains sodium and calcium chloride, the buffering agent HEPES does not substantially contribute to the formation of the fibrin hydrogel. This means that a fibrin hydrogel can be formed with the sodium and calcium salts in the absence of HEPES. Thus the concentration of HEPES in this example, as well as any other salt that does not substantially contribute to the formation of the fibrin hydrogel, is not included in the threshold concentration to form a fibrin hydrogel. High salt concentrations in the mixture can cause (e.g. dermal) tissue irritation and damage during the healing process as indicated by inflammatory cell infiltration as well as collagen degeneration and mineralization.

When the fibrin hydrogel salt (e.g. NaCl+CaCl₂) concentration was 0.423 wt.-% of the aqueous solution, a fibrin hydrogel may not be formed. Without intending to be bound by theory, it is believed that a salt (e.g. NaCl+CaCl₂) concentration of 0.423 wt.-% is insufficient to solubilize the fibrinogen. However, when the concentration of salt was greater than 0.423 wt.-%, a fibrin hydrogel readily formed. Hence, the threshold concentration to form a fibrin hydrogel is greater than 0.423 wt.-%. The threshold concentration of salt to form a gel is at least 0.430 wt.-% or 0.440 wt.-%, and in some embodiments at least 0.450, 0.500, 0.550, 0.600, 0.650, 0.700, 0.750, 0.800, 0.850, or 0.900 wt.-% of the aqueous solution. It is appreciated that the threshold concentration may vary to some extent depending on the selection of salt(s). The concentration of salt in the (i.e. initially formed) hydrogel is the same as the concentration of salt in the aqueous solution.

The present method comprises forming a mixture comprising of collagen and fibrin as previously described, and reducing the salt concentration below the threshold salt concentration to form a fibrin. In some embodiments, reducing the salt concentration below the threshold salt concentration to form a fibrin can occur before mixing fibrin and collagen to form a mixture comprising of collagen and fibrin. For embodiments wherein the (e.g. dehydrated) fibrin hydrogel is utilized for wound healing, the method comprises reducing the salt concentration below the concentration that can cause (e.g. dermal) tissue irritation and damage during the healing process.

In some embodiments, the step of reducing the salt concentration comprises rinsing the mixture of collagen-fibrin hydrogel or fibrin hydrogel with a solution capable of dissolving the salt. The solution is typically aqueous comprising at least 50, 55, 60, 65, 70, 75, 80, 85, 90, or 95 wt-%, or greater by volume water. The rinsing solution may further contain other water miscible liquids such as plasticizers. The collagen-fibrin hydrogel or fibrin hydrogel is typically rinsed with a volume of solution at least 1, 2, 3, 4, 5, 6, 7, 8, 9, or 10 times greater than the volume of the hydrogel. To reduce the salt concentration even further, the fibrin hydrogel may be rinsed with a volume of solution 11, 12, 13, 14, 15, 16, 17, 18, 19, or 20 times greater than the volume of the hydrogel. Another way of reducing the salt includes reacting the cation and/or anion of the salt, or in other words complexing the salt, such that the salt no longer forms ions in an aqueous solution such as bodily fluids of wounds. Another way of reducing the salt concentration is diluting with plasticizer. In some embodiments, reducing the salt concentration comprises dialyzing the mixture or the aqueous solution. Further, various combination of these methods can be used. The collagen-fibrin or fibrin hydrogel can be rinsed by immersion in rinse solution reservoir, with or without agitation, by spray washing, by rinsing under stream of wash solution, by percolating rinse through hydrogel, by dialysis or rinsing through more generally a separation membrane or fluid permeable membrane allowing rinse solution to flow through but not gel.

The amount of fibrin hydrogel forming salt (e.g. NaCl+CaCl₂) removed from the fibrin hydrogel can depend on the amount of salt in the aqueous (e.g. starting) solution or the mixture (collagen-fibrin hydrogel) and thus, the amount of salt in the initially formed hydrogel. For example, when the aqueous (e.g. starting) solution or the mixture comprises about 0.9 wt.-% salt, at least about 35 wt.-% of the salt is removed from the fibrin hydrogel or the mixture. However, when the aqueous (e.g. starting) solution or the mixture comprises about 1.25 wt.-% salt, greater than 50% of the salt is removed from the fibrin hydrogel or the mixture. In some embodiments, at least 40%, 45%, 50%, 55%, 60%, 65%, 70%, 75%, 80%, 85% or 90% of the salt is removed from the hydrogel or the mixture. In other embodiments, at least 91, 92, 93, 94, 95, 96, 97, 98, or 99% of the salt is removed from the hydrogel or the mixture. If the threshold concentration is less than 0.9 wt-%, the amount of salt removed can be less than 35 wt.-%. In such embodiment, at least 5%, 10%, 15%, 20%, 25%, 30%, 35%, 40%, or 45% of the salt is removed from the hydrogel or the mixture.

The mixture or gel of collagen and fibrin having the reduced fibrin hydrogel forming salt content can be then dehydrated using any number of methods. This step may be referred to as dehydrating, drying or desiccating, all of which refer herein to the process of removing water content from the mixture or gel as possible. Dehydration can therefore be accomplished using heat, vacuum, lyophilization, desiccation, filtration, air-drying, critical point drying, and the like. In some embodiments, lyophilization may be preferred since the resulting collagen-fibrin material is less likely to swell once in contact with an aqueous solution. However, the oven-dried gel sheets were observed to be more transparent and more uniform than the lyophilized sheets. The dehydration step may occur over a range of time, depending on the particular method used and the volume of mixture or gel. For example, the step may last for a few minutes, a few hours, or a few days. The present disclosure is not intended to be limited in this regard.

The dehydrated collagen-fibrin gel generally has a fibrin hydrogel forming salt concentration less than 30 wt.-% or 25 wt.-% for a water content no greater than 20 wt.-%. When dehydrated collagen-fibrin gel is intended for use for wound healing the salt concentration is less than 20, 19, 18, 17, 16, 15, 14, 13, 12, 11, 10, 9, 8, 7, 6, 5, 4, 3, 2, or 1 wt.-%, or less of the dehydrated collagen-fibrin gel having a water content no greater than 20 wt.-%. In some embodiments, the dehydrated collagen-fibrin gel has a water content no greater than 19, 18, 17, 16, 15, 14, 13, 12, 11, 10, 9, 8, 7, 6, 5, 4, 3, 2, or 1 wt.-% or less. In some embodiments, the total salt concentration including the buffering salts are also within the concentration ranges just described. In some embodiments, the dehydrated gel will swell when combined with water (i.e. rehydrated).

The dehydrated collagen-fibrin gel typically has a water content of at least 1, 2, 3, 4, or 5 wt-%. In some embodiments, the dehydrated collagen-fibrin gel has a water content of at least about 10, 15, or 20 wt-%.

The collagen-fibrin gel is dehydrated to reduce the water content and thereby increase the collagen-fibrin concentration. Higher collagen-fibrin concentrations generally promote healing more rapidly than lower collagen-fibrin concentrations. The collagen-fibrin gel, prior to dehydration typically comprises about 0.5 wt.-% to 5 wt.-% fibrin and 0.6 wt %-6 wt % collagen. After dehydration, the collagen-fibrin gel composition typically comprises at least 0.1, 10, 15, 20, 25, 30, 35, 40, 45, or 50 wt.-% collagen or fibrin. Fibrin or collagen concentration of the dehydrated gel is typically no greater than 99 wt.-% and in some embodiments no greater than 95, 90, 85, 80, 70, 60, 50, 40 or 30 wt.-%.

Since only a small concentration of fibrin-forming enzyme (e.g. thrombin) is needed to form fibrin and excess fibrin-forming enzyme (e.g. thrombin) is removed during rinsing, the concentration of fibrin-forming enzyme (e.g. thrombin) is also low in the dehydrated fibrin hydrogel. The dehydrated fibrin hydrogel typically includes fibrin-forming enzyme (e.g. thrombin) in an amount of thrombin no greater than 0.05 U/mg, or 0.005 U/mg, or 0.0005 U/mg, or 0.00005 U/mg. In some embodiments, the amount of fibrin-forming enzyme (e.g. thrombin) is 1 or 0.1 ppm relative to the concentration of fibrin.

The (e.g. dehydrated) collagen-fibrin gel can include various additives, provided the additives do not detract from forming the fibrin hydrogel and reducing the salt concentration therefrom. Examples of additives can include any of antimicrobial agents, anti-inflammatory agents, topical anesthetics (e.g., lidocaine), other drugs, growth factors, polysaccharides, glycosaminoglycans. If an additive is included, it should be included at a level that does not interfere with the activity of the collagen-fibrin containing layer with respect to promoting healing of the wound. Additional additives can include those listed in WO 2016/160541 (Bjork et al.), expressly incorporated herein by reference in their entirety into this disclosure.

The (e.g. dehydrated) fibrin or collagen-fibrin composition can have various physical forms, for example, hydrogel, a gel, a film, a paste, a sheet, a foam, or particles. In some embodiments, the fibrin hydrogel or collagen-fibrin hydrogel can be formed prior to reducing the salt content. The fibrin hydrogel or collagen-fibrin hydrogel is typically sufficiently flowable at a temperature ranging from 0° C. to 37° C. such that the fibrin hydrogel or collagen-fibrin hydrogel takes the physical form of the container surrounding the collagen-fibrin hydrogel. For, example if the fibrin hydrogel or collagen-fibrin hydrogel is cast into a rectangular pan, the fibrin hydrogel or collagen-fibrin hydrogel forms into a sheet. Thus, the fibrin hydrogel or collagen-fibrin hydrogel can be cast into various shaped containers or in other words molded to provide (e.g. dehydrated) hydrogel of various shapes and sizes.

In one embodiment, the (e.g. dehydrated) fibrin hydrogel or collagen-fibrin hydrogel may be provided as a foam. In another embodiment, the (e.g. dehydrated) fibrin hydrogel or collagen-fibrin hydrogel may be provided as particles. In other embodiments, the dehydrated fibrin hydrogel or collagen-fibrin hydrogel can be formed after reducing the salt content. For example, a sheet of (e.g. dehydrated) fibrin hydrogel or collagen-fibrin hydrogel can be (e.g. laser or die) cut into pieces having various shapes and sizes. In another example, the dehydrated hydrogel may be ground, pulverized, milled, crushed, granulated, pounded, and the like, to produce powder.

In other embodiments, (e.g. dehydrated) fibrin or collagen-fibrin composition can be admixed with natural or chemically modified and synthetic biological carrier materials. In typical embodiments, (e.g. dehydrated) fibrin hydrogel particles are provided on or within a carrier layer at a coating weight that is sufficient to provide the desired effect (e.g. promoting wound re-epithelialization). In some embodiments, the coating weight of the (e.g. dehydrated) fibrin hydrogel particles is typically at least 0.2, 0.5 or 1 milligram per cm² and typically no greater than 20, 10 or 5 milligrams per cm².

The conductivity determined by Method A of a solution containing 1% w/w (e.g. dehydrated) collagen-fibrin hydrogel composition described herein may be less than 7, 6, 5, 4, 3, 2, 1, 0.9, 0.8, 0.7, 0.6, 0.5, 0.4, 0.3, or 0.2 mS/cm. Method A is defined as follows: A 1% weight/volume suspension of the collagen-fibrin composition in purified water (18.2 megohm-cm at 25° C. water) was prepared. The water was maintained at 25° C. and the collagen-fibrin composition was completely immersed in the water. After immersion of the composition for at least 10 minutes, the conductivity of the water was measured in mS/cm using a conductivity meter.

In some embodiments, collagen-fibrin hydrogel composition may have a salt concentration less than 0.450, 0.400, 0.350, 0.300, 0.250, 0.200, 0.150, 0.100, 0.500, 0.400, 0.300, 0.200, 0.100 or 0.050 wt-%. In some embodiments, the fibrin of collagen-fibrin hydrogel composition may be a fibrin hydrogel. In some embodiments, the fibrin of collagen-fibrin hydrogel composition may be a fibrin hydrogel having a fibrin concentration ranging from 0.1 to 17 wt-%.

The (e.g. dehydrated) collagen-fibrin hydrogel composition described herein may be utilized as a wound dressing article. The wound dressing article described herein comprises a (e.g. dehydrated) collagen-fibrin composition in a suitable physical form such as a sheet (i.e. film), foam sheet, or collagen-fibrin disposed on or within a carrier layer. Thus, the (e.g. dehydrated) collagen-fibrin hydrogel layer can be provided in various forms as a continuous or discontinuous layer.

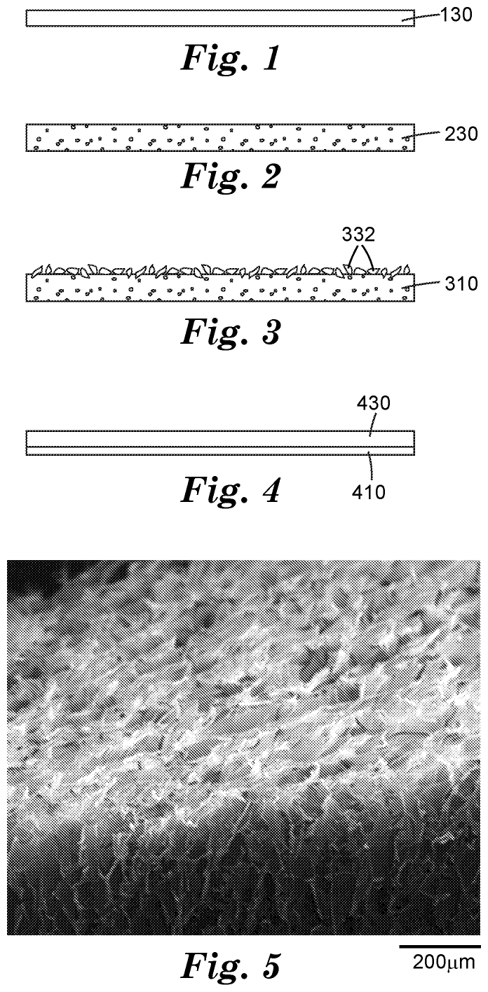

In some embodiments, the (e.g. dehydrated) collagen-fibrin hydrogel composition is formed prior to combining the (e.g. dehydrated) collagen-fibrin hydrogel composition with a carrier material or carrier layer. In other embodiments, a carrier layer is combined with the aqueous solution comprising collagen, fibrinogen, fibrin-forming enzyme (e.g. thrombin), and fibrin hydrogel forming salt or the fibrin hydrogel prior to reducing the salt and/or dehydration. For example, a fibrous (e.g. woven or nonwoven) substrate may be placed in a rectangular pan prior to adding the fibrin hydrogel thereby forming a sheet of collagen-fibrin hydrogel having a fibrous scrim embedded within the hydrogel. FIGS. 1-10 as follow illustrative some typical wound dressings articles.

FIG. 1 illustrates an embodiment of a collagen-fibrin article, suitable for use as a wound dressing. The collagen-fibrin article includes a (e.g. flexible) sheet 130 comprising or consisting of the (e.g. dehydrated) collagen-fibrin gel composition.

FIG. 2 illustrates another embodiment of a collagen-fibrin article, suitable for use as a wound dressing. The fibrin article includes a sheet of foam 230 comprising or consisting of the (e.g. dehydrated) collagen-fibrin gel composition. The foam may having various other shapes formed for example by molding the collagen-fibrin hydrogel composition (e.g. prior to hydrating) or cutting the foam into pieces after it is formed.

The collagen-fibrin sheet articles, such as illustrated in FIGS. 1 and 2 typically have a thickness of at least 10 μm, 15 μm or 20 μm and typically no greater than 5 mm, 4 mm, 3 mm, 2 mm, 1 mm, 0.5 mm, or 0.25 mm. In some embodiments, the thickness is no greater than 200, 150, 100, 75, or 60 microns. The basis weight of the collagen-fibrin sheet typically ranges from 2 to 10, 15, 20, 25 or 30 mg/cm².

The collagen-fibrin concentration of the sheet article is the same as the (e.g. dehydrated) collagen-fibrin hydrogel as previously described.

FIG. 3 illustrates another embodiment of a collagen-fibrin article, suitable for use as a wound dressing. The collagen-fibrin article includes a carrier sheet of collagen-fibrin sheet containing foam 230 or foam lacking collagen-fibrin 310. The foam 230 or 310 further comprises a plurality of collagen-fibrin particles 332 comprising the (e.g. dehydrated) collagen-fibrin hydrogel disposed on and/or within the pores of the wound-facing surface of the foam. The particles may be collagen-fibrin microbeads, collagen-fibrin microcarriers, or collagen-fibrin powder.

Each of the embodiments of FIGS. 1-3 may further comprise a carrier layer disposed on a major surface of the collagen-fibrin containing sheet article. A carrier layer is typically disposed on the opposing major surface as the wound-facing surface. For example, FIG. 4 illustrates an embodiment of a collagen-fibrin article, suitable for use as a wound dressing. The collagen-fibrin article includes a sheet 430 comprising or consisting of the (e.g. dehydrated) collagen-fibrin gel composition (e.g. 130, 230, or 310 together with 332) and a carrier layer 410.

In some embodiments, carrier layer 410 is a release liner. The release liner carrier may be disposed on the opposing major surface of both major surfaces (not shown) such that the fibrin-containing sheet is between the release liner layers.

Various release liners are known such as those made of (e.g. kraft) papers, polyolefin films such as polyethylene and polypropylene, or polyester. The films are preferably coated with release agents such as fluorochemicals or silicones. For example, U.S. Pat. No. 4,472,480 describes low surface energy perfluorochemical liners. Examples of commercially available silicone coated release papers are POLYSLIK™, silicone release papers available from Rexam Release (Bedford Park, Ill.) and silicone release papers supplied by LOPAREX (Willowbrook, Ill.). Other non-limiting examples of such release liners commercially available include siliconized polyethylene terephthalate films commercially available from H. P. Smith Co. and fluoropolymer coated polyester films commercially available from 3M under the brand “ScotchPak™” release liners.

In other embodiments, the carrier layer 410 may comprise a variety of other (e.g. flexible and/or conformable) carrier materials such as polymeric films and foams as well as various nonwoven and woven fibrous materials, such as gauze. In some embodiments, the carrier layer is absorbent, such as an absorbent foam. In other embodiments, the carrier layer is non-absorbent, such as a polymeric film.

The following embodiments are intended to be illustrative of the present disclosure and not limiting.

### Embodiments

Embodiment 1 is a method comprising mixing fibrin and collagen to form a mixture comprising of collagen and fibrin; and reducing the salt concentration below the threshold concentration to form a fibrin.
Embodiment 2 is the method of embodiment 1, wherein reducing the salt concentration below the threshold concentration to form a fibrin occurs before mixing fibrin and collagen to form a mixture.
Embodiment 3 is the method of embodiments 1 and 2, further comprising dissolving collagen or fibrin in acetic acid.
Embodiment 4 is the method of embodiments 1-3, wherein the fibrin is formed by mixing fibrinogen and fibrin-forming enzyme.
Embodiment 5 is the method of embodiments 1-4, wherein reducing the salt concentration comprises rinsing the mixture with an aqueous solution.
Embodiment 6 is the method of embodiments 1-5, wherein reducing the salt concentration comprises dialyzing the mixture.
Embodiment 7 is the method of embodiments 1-6, further comprising forming the mixture into a sheet, foam, or a plurality of pieces.
Embodiment 8 is the method of embodiments 1-7, further comprising dehydrating the mixture after reducing the salt concentration.
Embodiment 9 is the method of embodiments 1-8, wherein the dehydrating comprises freeze-drying, oven drying, critical point drying, or combination thereof.
Embodiment 10 is a collagen-fibrin hydrogel composition prepared by the method of embodiments 1-9.
Embodiment 11 is a method comprising mixing collagen, fibrinogen and fibrin-forming enzyme to form a mixture comprising of collagen and fibrin; and reducing the salt concentration below the threshold concentration to form a fibrin.
Embodiment 12 is a composition, comprising: a collagen and a fibrin; wherein the composition has a salt concentration below the threshold concentration to form a fibrin.
Embodiment 13 is the composition of embodiment 12, wherein the composition has a less than 7 mS/cm conductivity determined by Method A.
Embodiment 14 is the composition of embodiments 12-13, wherein the composition has a salt concentration less than 0.4 wt-%.
Embodiment 15 is the composition of embodiments 12-14, wherein the fibrin is a fibrin hydrogel having a fibrin concentration ranging from 0.1 to 17 wt-%.
Embodiment 16 is the composition of embodiments 12-15, further comprising a fibrin hydrogel forming salt.
Embodiment 17 is the composition of embodiment 16, wherein the fibrin hydrogel forming salt comprises calcium salt.
Embodiment 18 is the composition of embodiment 15, wherein the fibrin hydrogel is at least partially dehydrated.
Embodiment 19 is a wound dressing comprising a composition of embodiments 12-18.
Embodiment 20 is the wound dressing of embodiment 19, wherein the composition is disposed on or within a carrier.
Embodiment 21 is an article comprising a composition of embodiments 12-18.
Embodiment 22 is the article of the embodiment 21, wherein the article is in a form of sheet.
Embodiment 23 is the article of the embodiment 21, further comprising of a carrier layer.

### Examples

### Materials and Methods

Normal saline (0.9%) was prepared by dissolving 9 g of NaCl into 1 L of deionized water.
Purified water (18.2 megohm-cm at 25° C.) for conductivity testing was prepared using a MILLI-Q water purification system (EMD Millipore, Burlington, Mass.).
Lyophilizations were conducted using a VirTis Advantage Plus EL-85 Freeze Dryer (SP Scientific, Warminster, Pa.).
Dialysis tubes were prepared using FISHERBRAND Regenerated Cellulose Dialysis Tubing (15.9 mm diameter, 12,000-14,000 MW cut-off, Thermo Fisher Scientific, Waltham, Mass.) with each end of the tube sealed with a clamp.
Conductivity was measured using a VWR SYMPHONY conductivity meter (model B30PC1, VWR Corporation, Radnor, Pa.).
Calcium chloride and resazurin were obtained from the Sigma-Aldrich Company, St. Louis, Mo.
Collagen (type I from calf skin) was obtained from Elastin Products Company, Owensville, Mo.
Fibrinogen (bovine) was obtained from Rocky Mountain Biologics, Inc, Missoula, Mont.
Thrombin (bovine) was obtained from Cambryn Biologicals, LLC, Sarasota, Fla.

### Phosphate Buffered Saline pH 7.4 (PBS); Dulbecco's Modified Eagle Medium (DMEM); Pierce BCA

### Protein Assay Kits; Penicillin-Streptomycin (Pen-Strep) (10,000 U/mL); Fetal Bovine Serum (FBS); and

Human Dermal Fibroblasts, adult (HDFa) were obtained from Thermo Fisher Scientific, Waltham, Mass.
Serum supplemented DMEM was prepared by adding FBS and Pen-Step to DMEM to achieve a final concentration of 10% FBS and 1% Pen-Strep.
Fibrin particles were prepared by mixing thrombin (100 U with a 2.0 L solution of fibrinogen (10 mg/mL) in normal saline (0.9% NaCl) followed by incubating the mixture for 2-4 hours at 37° C. The resulting, fibrin gel was rinsed with deionized water and then homogenized using a blender. The homogenized fibrin slurry was poured into a tray (25.4 cm by 35.5 cm) and excess water was removed with a pipet. Next, the fibrin slurry was frozen at negative 80° C. for 2 hours and then lyophilized to provide the fibrin particles as a powder.
The resazurin solution was prepared from a 440 micromolar stock solution of resazurin in PBS. At the time of use the stock solution was diluted to a 44 micromolar solution by the addition of DMEM.

### EXAMPLES

### Example 1

A 9.6 mL solution of collagen (15 mg/mL) in acetic acid (20 mM) was combined with a 5.0 mL solution of fibrinogen (24 mg/mL) in normal saline (0.9% NaCl) and then a 0.14 mL solution of thrombin (100 U/mL) in normal saline (0.9% NaCl) was added with mixing to initiate the polymerization reaction. The resulting suspension was added to a dialysis tube. The filled dialysis tube was incubated at 37° C. for two hours and then immersed in a gently stirred bath of deionized water (2 L) at room temperature. The dialysis tube was maintained in the water bath overnight. Following the dialysis treatment, the resulting gel was cast into a plastic tray (inner diameter of 6.3 cm) that had been pre-treated with a food grade release agent and then frozen at negative 80° C. for at least one hour. The frozen sheet was lyophilized to provide the collagen-fibrin article as a solid, porous matrix. FIG. 5 is a Scanning Electron Microscopy (SEM) image of lyophilized collagen-fibrin article of Example 1.

### Example 2

A 9.6 mL solution of collagen (15 mg/mL) in acetic acid (20 mM) was combined with a 10.0 mL solution of fibrinogen (12 mg/mL) in normal saline (0.9% NaCl) and then a 0.14 mL solution of thrombin (100 U/mL) in normal saline (0.9% NaCl) was added with mixing to initiate the polymerization reaction. The resulting suspension was added to a dialysis tube. The filled dialysis tube was incubated at 37° C. for two hours and then immersed in a gently stirred bath of deionized water (2 L) at room temperature. The dialysis tube was maintained in the water bath overnight. Following the dialysis treatment, the resulting gel was cast into a plastic tray (inner diameter of 6.3 cm) that had been pre-treated with a food grade release agent and then frozen at negative 80° C. for at least one hour. The frozen sheet was lyophilized to provide the collagen-fibrin article as a solid, porous matrix.

### Example 3

A 6.7 mL solution of collagen (15 mg/mL) in acetic acid (20 mM) was combined with a 6.7 mL solution of fibrinogen (12 mg/mL) in normal saline (0.9% NaCl) and then a 0.14 mL solution of thrombin (100 U/mL) in normal saline (0.9% NaCl) was added with mixing to initiate the polymerization reaction. The resulting suspension was added to a dialysis tube. The filled dialysis tube was incubated at 37° C. for two hours and then immersed in a gently stirred bath of deionized water (2 L) at room temperature. The dialysis tube was maintained in the water bath overnight. Following the dialysis treatment, the resulting gel was cast into a plastic tray (inner diameter of 6.3 cm) that had been pre-treated with a food grade release agent and then frozen at negative 80° C. for at least one hour. The frozen sheet was lyophilized to provide the collagen-fibrin article as a solid, porous matrix.

### Example 4

A 13.4 mL solution of collagen (7.5 mg/mL) in acetic acid (20 mM) was combined with a 6.7 mL solution of fibrinogen (12 mg/mL) in normal saline (0.9% NaCl) and then a 0.14 mL solution of thrombin (100 U/mL) in normal saline (0.9% NaCl) was added with mixing to initiate the polymerization reaction. The resulting suspension was added to a dialysis tube. The filled dialysis tube was incubated at 37° C. for two hours and then immersed in a gently stirred bath of deionized water (2 L) at room temperature. The dialysis tube was maintained in the water bath overnight. Following the dialysis treatment, the resulting gel was cast into a plastic tray (inner diameter of 6.3 cm) that had been pre-treated with a food grade release agent and then frozen at negative 80° C. for at least one hour. The frozen sheet was lyophilized to provide the collagen-fibrin article as a solid, porous matrix.

### Example 5

A 13.4 mL solution of collagen (7.5 mg/mL) in aqueous CaCl₂) solution (1.25 M) was combined with a 6.7 mL solution of fibrinogen (12 mg/mL) in normal saline (0.9% NaCl) and then a 0.14 mL solution of thrombin (100 U/mL) in normal saline (0.9% NaCl) was added with mixing to initiate the polymerization reaction. The resulting suspension was added to a dialysis tube. The filled dialysis tube was incubated at 37° C. for two hours and then immersed in a gently stirred bath of deionized water (2 L) at room temperature. The dialysis tube was maintained in the water bath overnight. Following the dialysis treatment, the resulting gel was cast into a plastic tray (inner diameter of 6.3 cm) that had been pre-treated with a food grade release agent and then frozen at negative 80° C. for at least one hour. The frozen sheet was lyophilized to provide the collagen-fibrin article as a solid, porous matrix.

### Example 6

Collagen-fibrin articles were prepared according to the procedure described in Example 3 using different periods of dialysis treatment. Nine samples were prepared and submitted to a dialysis time of either 1, 2, 3, 4, 5, 6, 7, 8, or 20 hours. Following the prescribed dialysis time, each gel was cast into a plastic tray (inner diameter of 6.3 cm) that had been pre-treated with a food grade release agent and then frozen at negative 80° C. for at least one hour. The frozen sheet was lyophilized to provide the collagen-fibrin article as a solid, porous matrix.

Conductivity of each collagen-fibrin article was determined using the following METHOD A: A 1% weight/volume suspension of the collagen-fibrin article in purified water (18.2 megohm-cm at 25° C. water) was prepared. The water was maintained at 25° C. and the collagen-fibrin article was completely immersed in the water. After immersion of the article for at least 10 minutes, the conductivity of the water was measured in mS/cm using a conductivity meter. The results are reported in Table 1 and show that as dialysis time increased the measured conductivity was reduced. These results demonstrate that the salt concentration of the article can be lowered with increased dialysis time. The calculated salt content (weight %) of the collagen-fibrin article is also reported in Table 1. The calculated value was derived from the measured conductivity (assuming all of the dissolved solids being NaCl with a temperature of 25° C.). The measured conductivity was converted to parts per million (ppm) NaCl and then using a dilution factor of 100 converted to weight %.

TABLE 1

| | | Calculated salt |
|---|---|---|
| | | content of the |
| Dialysis | Conductivity | collagen-fibrin |
| Time (Hours) | (mS/cm) | article (weight %) |
| 1 | 3.92 | 21.8 |
| 2 | 3.26 | 18.0 |
| 3 | 1.92 | 10.4 |
| 4 | 1.22 | 6.4 |
| 5 | 0.76 | 3.8 |
| 6 | 0.68 | 3.4 |
| 7 | 0.42 | 1.9 |
| 8 | 0.18 | 0.5 |
| 20 | 0.14 | 0.3 |

### Example 7

A 5.0 mL solution of collagen (7.5 mg/mL) in acetic acid (20 mM) was mixed with a 5.0 mL solution of fibrinogen (6.24 mg/mL) in normal saline (0.9% NaCl) and a 5.14 mL solution of thrombin (2.7 U/mL) in normal saline (0.9% NaCl). The resulting suspension was added to a dialysis tube. The filled dialysis tube was incubated at 37° C. for two hours and then immersed in a gently stirred bath of deionized water (2 L) at room temperature. The dialysis tube was maintained in the water bath overnight. Following the dialysis treatment, the resulting gel was cast into a plastic tray (inner diameter of 6.3 cm) that had been pre-treated with a food grade release agent and then frozen at negative 80° C. for at least one hour. The frozen sheet was lyophilized to provide the collagen-fibrin article as a solid, porous matrix.

### Example 8

Lyophilized fibrin particles (80 mg, prepared as described above) were mixed into a 13.4 mL solution of collagen (7.5 mg/mL) in acetic acid (20 mM). The resulting solution was added to a dialysis tube. The filled dialysis tube was incubated at 37° C. overnight and then immersed in a gently stirred bath of deionized water (2 L) at room temperature. The dialysis tube was maintained in the water bath overnight. Following the dialysis treatment, the resulting gel was cast into a plastic tray (inner diameter of 6.3 cm) that had been pre-treated with a food grade release agent and then frozen at negative 80° C. for at least one hour. The frozen sheet was lyophilized to provide the collagen-fibrin article as a solid, porous matrix.

### Example 9

A solution was prepared of fibrinogen (16 mg/mL) and CaCl₂) (30 mM) in PBS. A 5.0 mL solution of collagen (20 mg/mL) in acetic acid (20 mM) was mixed with 5.0 mL of the fibrinogen solution and then 0.1 mL of thrombin (100 U/mL) was added. The resulting suspension was cast into a plastic tray (inner diameter of 6.3 cm) that had been pre-treated with a food grade release agent and then incubated at 37° C. for about two hours. The resulting gel was rinsed with 3 separate 25 mL portions of deionized water to remove inorganic salts. The gel was then dried at 60° C. until no residual water was observed to provide the collagen-fibrin article as a dry film.

### Example 10

A first solution was prepared of fibrinogen (20 mg/mL) and CaCl₂) (30 mM) in PBS. A second solution was prepared of CaCl₂) (30 mM) and glycerol (0.1%) in PBS. A 5.0 mL solution of collagen (20 mg/mL) in acetic acid (20 mM) was mixed with 4.0 mL of the first solution and 10.9 mL of the second solution. Next, 0.1 mL of thrombin (100 U/mL) was added. The resulting suspension was cast into a plastic tray (inner diameter of 6.3 cm) that had been pre-treated with a food grade release agent and then incubated at 37° C. for about two hours. The resulting gel was rinsed with 3 separate 25 mL portions of deionized water to remove inorganic salts. The gel was then dried at 60° C. until no residual water was observed to provide the collagen-fibrin article as a dry film.

### Example 11. Pro-Collagen I Expression: Fibroblasts Cultured in Media Conditioned with a Collagen-Fibrin Article

A portion of the collagen-fibrin article of Example 4 was immersed in DMEM at a concentration of 10 mg/mL. The suspension was incubated at 4° C. for 72 hours to condition the media. The conditioned liquid media was removed using a pipet and filtered through a 0.2 micron sterile filter (ACRODISC filter from Pall Corporation, Port Washington, N.Y.).

Human dermal fibroblasts, below passage 5, were seeded into a 24-well culture plate at a concentration of 50,000 cells/well in 1 mL of serum supplemented DMEM. The culture plate was incubated overnight (37° C., 5% CO₂ and 95% relative humidity). Next, the fibroblast cells were rinsed with 1 mL of DMEM (added and removed by pipet) and then cultured for 48 hours in 1 mL of the conditioned liquid media (37° C., 5% CO₂ and 95% relative humidity). As a negative control, fibroblast cells were cultured for 48 hours in 1 mL of fresh DMEM (37° C. and 5% CO₂ and 95% relative humidity).

At the completion of the culture period, the media was removed by pipet and the cells were rinsed with 1 mL of DMEM (added and removed by pipet). Fresh DMEM (1 mL) was added to each well and the cells were cultured for up to 48 hours (37° C., 5% CO₂ and 95% relative humidity). At time points of 1 hour and 24 hours, a portion (0.5 mL) of the culture supernatant was removed for determination of Pro-Collagen I expression. The supernatant was measured for total protein content with a Pierce BCA protein assay kit and the supernatant samples were normalized for protein content prior to testing with an ELISA human Pro-Collagen I assay kit (#ab210966 available from the Abcam Company, Cambridge, Mass.). In Table 2, the fold change for measured Pro-Collagen-I expression versus the negative control is reported.

### Example 12

The same procedure for determining Pro-Collagen I expression as described in Example 11 was followed with the exception that the collagen-fibrin article of Example 8 was used in place of the article from Example 4. The fold change for measured Pro-Collagen I expression versus the control was determined at 24 hour and 48 hour timepoints. The results are reported in Table 2.

TABLE 2

| Fibroblasts Cultured | | | |
|---|---|---|---|
| in Media Conditioned | Pro-Collagen I Expression Fold | | |
| with Collagen-Fibrin | Change from Negative Control | | |
| Article of | 1 hour | 24 hours | 48 hours |
| Example 4 | 4.3 | 2.6 | Not |
| | | | tested |
| Example 8 | Not | 8.1 | 3.4 |
| | tested | | |

### Example 13. Keratinocyte Growth Factor (KGF) Expression: Fibroblasts Cultured in Media Conditioned with a Collagen-Fibrin Article

A portion of the collagen-fibrin article of Example 8 was immersed in DMEM at a concentration of 10 mg/mL. The suspension was incubated at 4° C. for 72 hours to condition the media. The conditioned liquid media was removed using a pipet and filtered through a 0.2 micron sterile filter (ACRODISC filter).

Human primary fibroblasts, below passage 5, were seeded into a 24-well culture plate at a concentration of 50,000 cells/well in 1 mL of serum supplemented DMEM. The culture plate was incubated overnight (37° C., 5% CO₂ and 95% relative humidity). Next, the fibroblast cells were rinsed with 1 mL of DMEM (added and removed by pipet) and then cultured for 48 hours in 1 mL of the conditioned liquid media (37° C., 5% CO₂ and 95% relative humidity). As a negative control, fibroblast cells were cultured for 48 hours in 1 mL of fresh DMEM (37° C., 5% CO₂ and 95% relative humidity).

At the completion of the culture period, the media was removed by pipet and the cells were rinsed with 1 mL of DMEM (added and removed by pipet). Fresh DMEM (1 mL) was added to each well and the cells were cultured for 48 hours (37° C., 5% CO₂ and 95% relative humidity). At time points of 24 hour and 48 hours, a portion (0.5 mL) of the culture supernatant was removed for determination of KGF expression. The supernatant was measured for total protein content with a Pierce BCA protein assay kit and the supernatant samples were normalized for protein content prior to testing with an ELISA human KGF assay kit (#ab183362 available from the Abcam Company, Cambridge, Mass.). In Table 3, the fold change for measured KGF expression versus the negative control is reported.

TABLE 3

| | |
|---|---|
| Fibroblasts Cultured | |
| in Media Conditioned | KGF Expression Fold Change |
| with Collagen-Fibrin | from Negative Control |

| Article of | 24 hours | 48 hours |
|---|---|---|
| Example 8 | 1.6 | 2.4 |

### Example 14. Cell Viability: Fibroblasts Cultured in Media Conditioned with a Collagen-Fibrin Article

A portion of the collagen-fibrin article of Example 4 was immersed in DMEM at a concentration of 10 mg/mL. The suspension was incubated at 4° C. for 72 hours to condition the media. The conditioned liquid media was removed using a pipet and filtered through a 0.2 micron sterile filter (ACRODISC filter).

Human primary fibroblasts, below passage 5, were seeded into a 24-well culture plate at a concentration of 50,000 cells/well in 1 mL of serum supplemented DMEM. The culture plate was incubated overnight (37° C., 5% CO₂ and 95% relative humidity). Next, the fibroblast cells were rinsed with 1 mL of DMEM (added and removed by pipet) and then cultured for 48 hours in 1 mL of the conditioned liquid media (37° C., 5% CO₂ and 95% relative humidity). As a positive control, fibroblast cells were cultured for 48 hours in 1 mL of fresh serum supplemented DMEM.

At the completion of the culture period, the media was removed by pipet and the cells were rinsed with 1 mL of DMEM (added and removed by pipet). Fresh DMEM (1 mL) was added to each well and the cells were cultured for 48 hours (37° C., 5% CO₂ and 95% relative humidity). Next, the DMEM was removed by pipet from each well and replaced with 1 mL of the resazurin solution (44 micromolar). The culture plate was incubated at 37° C. for 4 hours. Following incubation, a 100 microliter sample of the media was removed from each well and transferred to a 96-well plate. Absorbance measurements were taken at 560 nm and 600 nm with an INFINITE M200 microplate reader (Tecan Group Ltd., Mannedorf, Switzerland). The measured absorbance values were normalized by subtracting the measured absorbance of resazurin only solution at 600 nm from the measured absorbance of samples at 560 nm. The percent viability results (mean of 3 replicates) are reported in Table 4 as a percentage of normalized positive control.

### Example 15. Cell Viability: Fibroblasts Cultured in Media Conditioned with a Collagen-Fibrin Article

The same procedure for determining cell viability as described in Example 14 was followed with the exception that the collagen-fibrin article of Example 8 was used in place of the article from Example 4. The percent viability results (mean of 3 replicates) are reported in Table 4.

TABLE 4

| Fibroblasts Cultured | |
|---|---|
| in Media Conditioned | |
| with Collagen-Fibrin | |
| Article of | Percent Viability |
| Example 4 | 90% |
| Example 8 | 90% |

1. A method comprising
mixing fibrin and collagen to form a mixture comprising of collagen and fibrin; and
reducing the salt concentration below the threshold concentration to form a fibrin.

2. The method of claim 1, wherein reducing the salt concentration below the threshold concentration to form a fibrin occurs before mixing fibrin and collagen to form a mixture.

3. The method of claim 1, further comprising dissolving collagen or fibrin in acetic acid.

4. The method of claim 1, wherein the fibrin is formed by mixing fibrinogen and fibrin-forming enzyme.

5. The method of claim 1, wherein reducing the salt concentration comprises rinsing the mixture with an aqueous solution.

6. The method of claim 1, wherein reducing the salt concentration comprises dialyzing the mixture.

7. The method of claim 1, further comprising forming the mixture into a sheet, foam, or a plurality of pieces.

8. The method of claim 1, further comprising dehydrating the mixture after reducing the salt concentration.

9. The method of claim 1, wherein the dehydrating comprises freeze-drying, oven drying, critical point drying, or combination thereof.

10. A collagen-fibrin hydrogel composition prepared by the method of claim 1.

11. A method comprising
mixing collagen, fibrinogen and fibrin-forming enzyme to form a mixture comprising of collagen and fibrin; and
reducing the salt concentration below the threshold concentration to form a fibrin.

12. A composition, comprising:
a collagen and
a fibrin;
wherein the composition has a salt concentration below the threshold concentration to form a fibrin.

13. The composition of claim 12, wherein the composition has a less than 7 mS/cm conductivity determined by Method A.

14. The composition of claim 12, wherein the composition has a salt concentration less than 0.4 wt-%.

15. The composition of claim 12, wherein the fibrin is a fibrin hydrogel having a fibrin concentration ranging from 0.1 to 17 wt-%.

16. The composition of claim 12, further comprising a fibrin hydrogel forming salt.

17. The composition of claim 16, wherein the fibrin hydrogel forming salt comprises calcium salt.

18. The composition of claim 15, wherein the fibrin hydrogel is at least partially dehydrated.

19. A wound dressing comprising a composition of claim 12.

20. The wound dressing of claim 19, wherein the composition is disposed on or within a carrier.
